SEQUENCE LISTING (4 sequences)

SEQ ID NO: 1 - protein, 123 aa, synthetic construct
KQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 2 - protein, 124 aa, synthetic construct
MKQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 3 - protein, 39 aa, Homo sapiens
KQFTKXELSQLLKDIDGYGGIALPELIXTMFHTSGYDTQ

SEQ ID NO: 4 - protein, 43 aa, Homo sapiens
LDDDITDDIMXAKKILDIKGIDYWLAHKALXTEKLEQWLXEKL

(19) United States
(12) Patent Application Publication
(10) Pub. No.: US 2026/0242874 A1
(43) Pub. Date: Aug. 20, 2026

(54) METHOD OF PREDICTING RESPONSE TO TREATMENT

(71) Applicant: LINNANE PHARMA AB, Malmö (SE)

(72) Inventors: Catharina SVANBORG, Lund (SE); Siddharth CHINCHANKAR, Lund (SE); Farhan HAQ, Lund (SE)

(73) Assignee: LINNANE PHARMA AB, Malmö (SE)

(21) Appl. No.: 19/161,523

(22) PCT Filed: Mar. 4, 2024
(86) PCT No.: PCT/EP2024/055619

§ 371 Date: Sep. 2, 2025

(30) Foreign Application Priority Data
Mar. 3, 2023 (GB) 2303177.6

(51) Int. Cl. C12Q 1/6886

(52) U.S. Cl. CPC C12Q 1/6886; C12Q 2600/106; C12Q 2600/158

(57) ABSTRACT

The invention provides a method of predicting the outcome of treatment of cancer or a metabolic-related condition in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of cancer genes in the subject following administration of the complex, and b) correlating the regulation of the genes to the outcome of the treatment, wherein a positive outcome of treatment is inversely related to the level of regulation of those genes.

### TECHNICAL FIELD

The present invention relates to a method of predicting a response to treatment, particularly treatment of cancer using a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt.

### BACKGROUND

HAMLET (human alpha-lactalbumin made lethal to tumor cells) is the first member of a family of tumoricidal unfolded protein-lipid complexes, consisting of partially unfolded α-lactalbumin and oleic acid. Initially isolated in the form of a fraction obtained by passing a casein containing fraction of human milk down an ion exchange column under high salt conditions (WO96/004929), it was found to be biologically active and in particular had an antibacterial activity. Subsequently, other methods for preparing active complexes have been derived including methods in which α-lactalbumin from various sources and oleic acid are heated together in solution. In addition, however, HAMLET and related complexes such as BAMLET, derived from bovine alpha-lactalbumin, have been found to kill transformed cells such as tumour cells or papilloma cells, as well as having antiviral activity. HAMLET kills many types of tumor cells in vitro and this tumoricidal activity is maintained in vivo, as shown in animal models of human glioblastoma xenografts and bladder cancer. Topical application of HAMLET removed or reduced skin papillomas and local instillations of HAMLET killed bladder cancer cells but not healthy cells in surrounding tissues and caused a reduction in tumor size. The sensitivity of tumor cells to HAMLET reflects oncogenic transformation and is modified by the glycolytic state of the cell (Storm P, et al. (2011). Oncogene). shRNA silencing of c-Myc or Ras pathway members conferred resistance to HAMLET and the level of c-Myc expression paralleled HAMLET sensitivity. Furthermore, glucose deprivation sensitized tumor cells to HAMLET and the HAMLET-sensitivity was modified by shRNAs targeting glycolytic enzymes. Additionally, HAMLET was shown to have pronounced effects on global metabolism with a rapid metabolic paralysis in tumor cells and potential diversion of the glycolytic flux towards the pentose phosphate pathway.

Complexes such as HAMLET have been demonstrated previously as being therapeutic in the treatment of a range of pre-existing cancers (WO2005/082406), and for the prophylactic treatment of colon cancer (WO2014/023976). The inventors have also recently shown that such complexes are useful in the treatment of cancers to which the complexes cannot be directly applied (e.g., peroral application for cancers outside the GI tract) and for the treatment of metabolic-related conditions.

The inventors have now identified that the response to treatment with the complexes is surprisingly rapid and easily identifiable. Remarkably, it is possible to assess very quickly which subjects are responding well to treatment, and those that are responding less well and may benefit from alternative or additional treatments such as surgery. This is a very important finding as it will allow subjects that are responding well to avoid surgery, and to ensure that subjects in need of additional treatment receive it as soon as possible.

### SUMMARY OF THE INVENTION

The invention provides a method of predicting the outcome of treatment of cancer in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of cancer genes in the subject following administration of the complex, and b) correlating the regulation of the genes to the outcome of the treatment, wherein a positive outcome of treatment is inversely related to the level of regulation of those genes.

Also provided is a method of predicting the outcome of treatment of a metabolic-related condition in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of cancer genes in the subject following administration of the complex, and b) correlating the regulation of the genes to the outcome of the treatment, wherein a positive outcome of treatment is inversely related to the level of regulation of those genes.

### The Cancer

The cancer may be any cancer. For example, it may be found in one or more of the nasal passage, the GI tract (e.g., in one or more of the oral cavity, the stomach, the colon, the bowel), the central nervous system (e.g., in the brain, the spine), the lung, the kidney, the vagina, the bladder, the liver, the skin, the breast, the prostate and/or the ovary. In particular, the cancer may be found in bladder. The cancer may be a primary cancer or a metastasis.

### The Metabolic-Related Condition

The metabolic-related condition may be any condition that is impacted by, or has an impact on metabolism. For example, it may be one or more of insulin resistance, type II diabetes, metabolic syndrome, non-alcoholic fatty acid liver disease, cirrhosis, high blood pressure. It may be a condition arising from challenges with processes such as moderation or modulation of insulin tolerance or sensitivity, lipid metabolism and/or glucose metabolism.

Such metabolic conditions may be related to the presence of cancer, for example they may be secondary to cancer, or may be independent thereof.

### The Complex

The complex comprises a polypeptide having a sequence of a naturally occurring alpha-lactalbumin, or a functional variant thereof; or a peptide of up to 50 amino acids comprising an alpha-helical domain of said polypeptide, and a fatty acid or lipid or salt thereof.

In one embodiment, the polypeptide has a sequence of a naturally occurring alpha-lactalbumin, preferably a human or bovine alpha-lactalbumin, more preferably a bovine alpha-lactalbumin.

In one embodiment, the alpha-helical domain is the Alpha 1 (residues 1-39) or Alpha 2 (residues 81-123) domain of human alpha-lactalbumin, being of of SEQ ID NO 3 or SEQ ID NO 4; KQFTK XELSQLLKDIDGYGGIALPELI XTMFHTSGYDTQ (SEQ ID NO 3) LDDDITDDIM XAKKILDIKGIDYWLAHKALXTEKLEQWL XEKL (SEQ ID NO 4) where X is an amino acid residue other than cysteine.

In an embodiment, the complex comprises a peptide of about or less than 45, 42, or 40 amino acids, in particular 39 amino acids, preferably corresponding to the Alpha 1 domain of human alpha-lactalbumin.

In one embodiment, the functional variant consists of a sequence lacking disulfide bonds. In one embodiment, the functional variant consists of a sequence in which cysteine residues in the native alpha-lactalbumin are changed to other amino acid residues, preferably alanine residues.

In one embodiment, the fatty acid or lipid or salt thereof is a fatty acid or salt thereof. In one embodiment, the fatty acid or salt thereof is oleic acid or an oleate salt.

In one embodiment, the polypeptide has the sequence of bovine alpha-lactalbumin and the fatty acid or salt thereof is oleic acid or an oleate salt.

The polypeptide present in the complex may have the sequence of an α-lactalbumin or a variant thereof as described above.

The complex may be referred to as a biologically active complex. As used herein, the term “biologically active” means that the complex has a biological activity, which is different from, or stronger than the individual components. In particular, the complex is able to induce cell death in particular selectively in tumour cells and/or has a bactericidal or antiviral effect not seen with the native protein including for example monomeric α-lactalbumin forms, although other therapeutic effects may be available.

The expression “variant” refers to proteins or polypeptides having a similar biological function but in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt the function of the DNA binding domain polypeptides. Broadly speaking, fewer non-conservative substitutions will be possible without altering the biological activity of the polypeptides.

Determination of the effect of any substitution (and, indeed, of any amino acid deletion or insertion) is wholly within the routine capabilities of the skilled person, who can readily determine whether a variant polypeptide retains the fundamental properties and activity of the basic protein. For example, when determining whether a variant of the polypeptide falls within the scope of the invention, the skilled person will determine whether complexes comprising the variant retain biological activity (e.g., tumour cell death) of complexes formed with unfolded forms of the native protein and the polypeptide has at least 60%, preferably at least 70%, more preferably at least 80%, yet more preferably 90%, 95%, 96%, 97%, 98%, 99% or 100% of the native protein.

Variants of the polypeptide may comprise or consist essentially of an amino acid sequence with at least 70% identity, for example at least 75%, 80%, 85%, 90%, 91%, 92%, 93%, 94%, 96%, 97%, 98% or 99% identity to a native protein sequence such as an alpha-lactalbumin or lysozyme sequence.

The level of sequence identity is suitably determined using the BLASTP computer program with the native protein sequences as the base sequence. This means that native protein sequences form the sequence against which the percentage identity is determined. The BLAST software is publicly available at http://blast.ncbi.nlm.nih.gov/Blast.cgi (accessible on 12 Mar. 2009).

In a particular embodiment, the polypeptide is an α-lactalbumin such as human, bovine or ovine α-lactalbumin. Whilst variants of these as described above may be useful in the invention, for nutraceutical use in particular, it may be preferable to utilize the native proteins in the products. A particular embodiment used human α-lactalbumin. In another embodiment, the α-lactalbumin is bovine α-lactalbumin. The sequence of a wide range of α-lactalbumins is known in the literature, for example as shown in Watanabe et al., J. Vet Med Sci, (2000) 62(11); 1217-1219.

In another embodiment, the polypeptide comprises a recombinant protein having the sequence of α-lactalbumin or a fragment thereof but which lacks intra-molecular disulfide bonds or cross-links. By ensuring that the recombinant protein lacks intra-molecular disulfide crosslinks, the molecule will be three-dimensionally non-native and completely inactive in terms of its original endogenous biological activity. This is achieved by changing cysteine residues in the native α-lactalbumin to other residues, in particular alanine residues. Preferably all cysteine residues will be changed to other residues, such as alanine residues. In particular the recombinant protein is based upon the sequence of human α-lactalbumin but α-lactalbumin from other sources, including bovine or ovine α-lactalbumin may be used to derive the recombinant protein.

In a particular embodiment, the polypeptide is a recombinant protein having the sequence of native mature α-lactalbumin but which has all of the cysteines found at positions 6, 28, 61, 73, 77, 91, 111 and 120 in the full length sequence of mature human α-lactalbumin mutated to other amino acids, such as alanine, which do not give rise to disulphide bridges. Thus, a particular of a protein that may be utilised in accordance with the invention comprises a protein of SEQ ID NO 1.

(SEQ ID NO 1)
KQFTKAELSQLLKDIDGYGGIALPELIATMFHTSGYDTQAIVEN
NESTEYGLFQISNKLWAKSSQVPQSRNIADISADKFLDDDITDD
IMAAKKILDIKGIDYWLAHKALATEKLEQWLAEKL

where the bold type indicates positions of mutations of cysteines in native human α-lactalbumin.

As reported in WO2010079362, additional amino acid residues, for example up to 20 amino acids, may be attached at N and/or C terminal of the protein, if convenient, for example for expression purposes. Thus in particular, a recombinant protein as shown in SEQ ID NO. 1 but with an additional methionine at the N-terminus (SEQ ID NO 2 shown below) has been used in the complex of the invention.

(SEQ ID NO 2)
MKQFTKAELSQLLKDIDGYGGIALPELIATMFHTSGYDTQAIVE
NNESTEYGLFQISNKLWAKSSQVPQSRNIADISADKFLDDDITD
DIMAAKKILDIKGIDYWLAHKALATEKLEQWLAEKL

The polypeptide used in the complex is suitably in pure form, and is suitably prepared using conventional methods of peptide synthesis or by recombinant expression. In particular, DNA encoding the required recombinant α-lactalbumin can be inserted into suitable expression vectors such as plasmids, which can then be employed to transform host cells, for example, prokaryotic cells such as E. coli or eukaryotic cells such as particular insect cells using conventional methods.

Suitable fatty acids or lipids include those known to provide biologically active complexes. These include fatty acids, for example as described in WO2008058547. Where salts are used, these are suitably water soluble salt. Particular examples of suitable salts may include alkali or alkaline earth metal salts. In a particular embodiment, the salt is an alkali metal salt such as a sodium- or potassium salt. Where used in pharmaceuticals, the salts will be pharmaceutically acceptable.

Particular examples of fatty acids or lipids used in the present invention are those having from 4-30, for example from 6 to 28, such as from 8 to 26 carbon atoms. In particular embodiments, the fatty acid or lipid has from 10 to 24, such as from 12 to 22, for example from 14 to 20 carbon atoms. In particular, the fatty acid or lipid will have 16, 17, 18 or 20 carbon atoms. The fatty acids may be saturated or unsaturated.

In particular however, the complexes of the invention utilize fatty acids or salts of fatty acids having 18 carbon atoms. In one embodiment, the complexes of the invention utilize fatty acids or salts of fatty acids having 18 carbon atoms and wherein the fatty acid chain is unsaturated. In one embodiment, the fatty acid or salt of the fatty acid is a C18:1 fatty acid or salt thereof. A specific example is a C18:1 fatty acid or salt thereof of formula CH₃(CH₂)₇CH═CH(CH₂)₇COOH or CH₃(CH₂)₇CH═CH(CH₂)₇COO⁻. In one embodiment, the fatty acid or salt thereof is oleic acid or oleate salt.

The complex may be prepared using methods similar to those described for example in WO99/26979, WO2008/138348, WO2010/131237, WO2014/023976, WO2018/210759, and WO2022/073982 the content of which is incorporated herein by reference. Not only has it been found that complexes can be prepared by contacting unfolded α-lactalbumin or derivatives thereof with co-factors in particular oleic acid or salts thereof under ion exchange conditions such as those found on an ion exchange column, but also incubation of solutions of α-lactalbumin or derivatives thereof with a co-factor at elevated temperatures, for example of from 50-80° C., for example from 50-70° C. and in particular between 55-60° C. will result in the production of suitable complexes for use in the invention.

These methods however have generally focused on attempting to recreate the conditions in which the protein becomes unfolded and complexed with oleic ions. Such work has focused on using pure proteins including recombinant variant versions of the base proteins to facilitate the production of active complexes. Such starting materials however can also increase the cost of production.

It is known that complexes obtained using α-lactalbumin from sources other than human milk, and in particular, BAMLET, obtained using bovine α-lactalbumin shows a qualitatively similar effects on cells and in particular on tumour cells as HAMLET (see for instance, Rammer et al. (2010) Mol. Cancer Ther. 9(1) 24-32). Therefore, effects demonstrated hereinafter using BAMLET would be similarly observed if HAMLET or compositions based upon HAMLET are used instead of BAMLET.

### The Treatment

The complex may have been administered via any appropriate route and in any appropriate doses.

### The Method of Predicting the Outcome of the Treatment

The method may be carried out using any appropriate steps to assess the regulation of cancer genes. For example, it may comprise measuring one or more markers of cancer gene regulation in a sample obtained from the subject.

The one or more markers may be any marker of cancer gene regulation, such as a genetic or molecular marker. In particular, the marker may be DNA or RNA, especially RNA.

The sample may be any sample in which cancer gene regulation can be measured. It may, for example, be a sample of urine, saliva, blood, cerebrospinal fluid (CSF), menstrual blood, cervicovaginal fluid, semen, synovial fluid, pleural lavage, pericardial fluid, amniotic fluid, nasal fluid, otic fluid, gastric fluid or feces. It is typically a sample of a bodily fluid. In an embodiment, it is a urine sample.

Ideally, the sample is taken from or close to the site of the cancer being treated, but it may be remote from the cancer being treated. For example, if the cancer is bladder cancer, the sample may be a urine sample. If the cancer is a cancer of the oral cavity, the sample may be saliva. If the cancer is a cancer of the central nervous system, the sample may be CSF.

A significant number and array of genes are regulated by treatment with the complex. Regulation may mean upregulation or downregulation or inhibition. The method may comprise assessing both up and down regulation of cancer genes.

Genes downregulated, or inhibited by the complex include, but are not limited to genes associated with angiogenesis, such as ADIPOQ, CCL11, CCL2, CCN1, CSF3, CXCL1, CXCL2, CXCL6, CXCL8, DCN, EGR3, EREG, FGF7, FPR2, GREM1, HBEGF, HTRA1, LAMA2, LEP, LYVE1, MEOX2, MMP3, NTRK2, PROKR1, PTGS1, PTN, PTPRZ1, PTX3, RSPO3, SELE, SFRP1, SFRP2, SVEP1, TF, TNMD and VEGFD; genes associated with tumor cell movement and migration, such as CHD5, CNN1, CXCL3, EFEMP1, FLNC, GC, GREB1, KLF9, NNMT, XIST, EYA1, SHC4 and TNS1; genes associated with metastasis such as CTHRC1, EPHA6, FBN2, P2RY6, PDLIM3, PLCXD3, PPP2R2C, PRAME, PTPRT and SERPINB2; genes associated with cell survival such as APOD, CALB1, CRH, DDX43, DGKB, DUSP1, GADD45B, LGALS7/LGALS7B, MFAP5, PDK4, PLAGL1, TFPI2 and ZBTB16; and genes associated with inflammatory response such as CNTNAP2, DUOXA2 and ULBP1.

In an embodiment, the method comprises measuring the regulation of one, two, three, four, five, six, seven, eight, nine, ten, 15, 20 or 25 or more genes associated with one or more of angiogenesis, tumor cell movement and migration, metastasis, cell survival, inflammatory response, organismal death, cell movement, migration of cells, organization of cytoskeleton, cell movement of tumor cell lines, activation of blood cells, activation of cells, development of neurons, leukocyte migration and/or chemotaxis.

Additionally or alternatively, the method comprises measuring the regulation of one, two, three, four, five, six, seven, eight, nine, ten, 15, 20 or 25 or more genes associated with one or more of the following pathways: S100 Family Signalling Pathway, Phagosome Formation, Pathogen Induced Cytokine Storm Signalling Pathway, FAK Signalling, CREB Signalling in Neurons, Breast Cancer Regulation by Stathmin1, Cardiac Hypertrophy Signalling (Enhanced), G-Protein Coupled Receptor Signalling, IL-17 Signalling and Pulmonary Fibrosis Idiopathic Signaling Pathway.

In an embodiment, the method comprises measuring the regulation of one, two, three, four, five, six, seven, eight, nine, ten, 15, 20 or 25 or more cancer genes, especially genes regulated by the administration of the complex, the genes comprising one, two, three, four, five, six, seven, eight, nine, ten or more genes selected from ADIPOQ, CALB1, EREG, CXCL2, CXCL6, GC, CSF3, PTPRZ1, DGKB, CRH, XIST, SLC5A1, CCL11, NEGR1, CLDN10, TF.

The method is carried out on a subject to which the complex has been administered or on a sample obtained from that subject. The method may generally be carried out, and/or the sample is generally obtained within 30, 45, 60, 75, 90, 105, 120, 135, 150 minutes, 4, 5, 6, 7, 8, 9, 10, 11, 12, 14, 16, 18, 20, 22, 24, 36, 48, 60, 72, 84, 96, 108 or 120 hours of the administration of the complex.

If the complex is administered over a period of time, the method may generally be carried out, and/or the sample is generally obtained within 1, 2, 3, 4, 5, 6, 7, 8, 9, 10, 11, 12, 14, 16, 18, 20, 22, 24, 36, 48, 60, 72, 84, 96, 108 or 120 hours of completion of the administration of the complex.

If the complex is administered more than once, the method may generally be carried out and/or the sample is generally obtained within 1, 2, 3, 4, 5, 6, 7, 8, 9, 10, 11, 12, 14, 16, 18, 20, 22, 24, 36, 48, 60, 72, 84, 96, 108 or 120 hours of the first or subsequent or last administration of the complex.

Correlation with Cancer

The complex regulates a large number of cancer genes. The inventors have found that ongoing or high levels of regulation of those genes following administration of the complex is indicative of the continuing presence of cancer cells, which generally leads to a less successful outcome of treatment. On the other hand, lower levels of cancer gene regulation or the absence of regulation are indicative that fewer cancer cells remain and that treatment with the complex has been more or completely successful. In other words, a high level of gene regulation is associated with a less successful outcome, i.e., the cancer is responding somewhat to treatment, but further treatment such as surgery is likely to be required. A low level of level of gene regulation is associated with a more successful outcome, i.e., the cancer is responding well to treatment, and further treatment such as surgery should not be required.

Correlation with Metabolic-Related Conditions

The inventors have also found that ongoing or high levels of regulation of genes in the ADIPOQ network, such as those shown in FIGS. 3A and 3B, following administration of the complex is indicative of a less successful outcome of treatment. On the other hand, lower levels of gene regulation or the absence of regulation is indicative that treatment with the complex has been or will be more or completely successful.

### Methods of Treatment

The invention also provides a method of treating cancer, comprising administering a therapeutic amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to a subject; assessing the regulation of cancer genes in the subject following administration of the complex, correlating the regulation of the genes to the outcome of the treatment, wherein a positive outcome of treatment is inversely related to the level of regulation of those genes, and determining and/or delivering a further course of treatment.

The further course of treatment may comprise administering another therapeutic agent, or surgical intervention, or both.

The invention also provides a method of treating a metabolic-related condition, comprising administering a therapeutic amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to a subject; assessing the regulation of cancer genes in the subject following administration of the complex, correlating the regulation of the genes to the outcome of the treatment, wherein a positive outcome of treatment is inversely related to the level of regulation of those genes, and determining and/or delivering a further course of treatment.

The further course of treatment may comprise administering another therapeutic agent, or surgical intervention, or both.

### General

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows differential gene expression in 14 individual tumors treated with 8.5 mM of alpha1-oleate.
(a) Individual tumors in patients treated with 8.5 mM of alpha1-oleate were defined as complete responders (CR), if the tumor was removed, partial responders (PR) if the tumor size was reduced, stable disease if the tumor size was unchanged (SD) or non-responders, if the tumor size increased. Cut off 25% change in tumor size. (b) Gene expression relative to outcome defined by three-dimensional Principal Component Analysis (PCA) of 14 individual tumors (Green=SD, Blue=PR and Red=CR). The majority of CR and PR tumors are closely clustered with low variance. The four SD tumors are clustered independently, at a distance from te the responding tumors. (c) Gene expression in individual tumors distinguishing CR, PR and SD groups (red=upregulated, blue=downregulated), with a predominance of inhibited genes (z scores, compared to PR and CR tumors). (d) The top regulated canonical pathways in SD, PR and CR tumors sorted by z-scores (blue=inhibition\z-score<−2 and orange=activation\z-score>2). Pathway analysis detected a stronger response to alpha1-oleate was detected in patients with remaining tumor tissues with a profile of inhibition of S100 Family signaling, phagosome formation, cytokine storm signaling, FAK signaling and CREB signaling pathway is observed in SD tumors compared to PR and CR tumors. (e) The top regulated biofunctions in SD, PR and CR tumors were sorted by z-score (blue=inhibition\z-score<−2 and orange=activation\z-score>2). Biofunction analysis detected a strong inhibition of cell activation, movement, migration and activation of organismal death can be seen in SD tumors compared to PR and CR tumors. (f) Functional classification of the shared, strongly inhibited genes (z-score<−2, P<0.05). Top inhibited functions include genes related to angiogenesis, tumor cell survival, movement, migration, metastasis and inflammation. (g) Top inhibited Genes In Individual Tumors, showing significant inhibition in SD, compared with lower and lowest inhibition in PR an CR tumors.

Patients with non-muscle invasive bladder cancer received intra-vesical instillations of alpha1-oleate.

FIG. 2 shows the results of gene expression analysis from subjects treated with the complex v. those treated with a placebo.

FIG. 3 shows up and down-regulation of genes in the ADIPOQ Network following treatment with 8.5 mM complex (a) and 1.7 mM complex (b).

FIG. 4 shows adiponectin protein levels in patients treated with the complex vs. placebo.

FIGS. 5 and 6 show adiponectin receptor and complex interaction. FIG. 5C shows an overlay of AlphaFold predicted binding of Alpha1 peptide to ADIPOR1 and ADIPOR2 demonstrating high structural homology between AdipoR1 and AdipoR2, and a common binding pocket for the Alpha1 peptide in both receptor proteins.

### EXAMPLES

### Effects on Tumor Number, Size and Appearance

### Changes in Individual Tumors Recorded by Endoscopy.

Macroscopic changes in tumor number or tumor size were recorded by endoscopy and camera, using a flexible cystoscope with white light- and narrow band imaging. Changes in tumor size were estimated by comparing the tumor at the time of diagnosis and post treatment, prior to transurethral resection of bladder tumor. The fully opened clamps of the flexible forceps were used as a measuring devise.

Significant changes were observed in treated tumors, compared to the placebo group. Treatment was associated with changes in tumor number and tumor size.

### Tumor Histopathology

The tumor response was further evaluated by histopathology. Significant changes were observed in treated tumors, compared to the placebo group. A complete response was confirmed by a lack of tumor tissue in the control biopsy or presence of minor residual tissue stroma without tumor cells. In the partial responders, fragmentation and cell shedding left few intact papilli and the tissue surface appeared denuded. In addition, tumor tissue surrounding the target lesion was affected in the responding patients, including satellite tumors, which had disappeared.

### Immediate Tumor Response

Intravesical alpha1-oleate instillations triggered a rapid tumor response, which was quantified by comparing the pre-instillation sample in each patient to the first urine sample obtained post instillation (≤2 hours).

### Cell Shedding.

Cell shedding was detected in all patients, suggesting a direct and rapid effect of alpha1-oleate on their tumors. Intravesical alpha1-oleate instillations triggered rapid tumor cell shedding into the urine. A highly significant increase in cell numbers was detected post instillation at each visit compared to each pre-instillation sample.

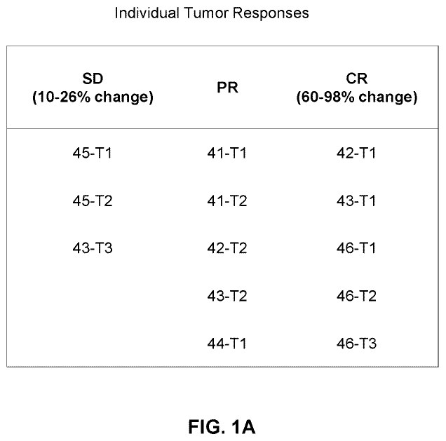

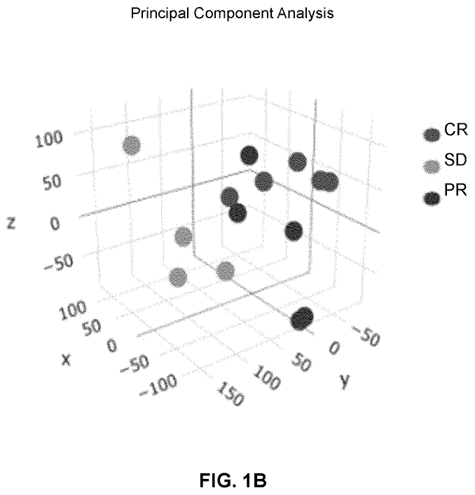

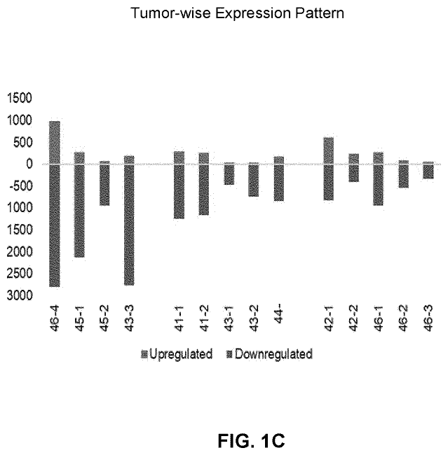

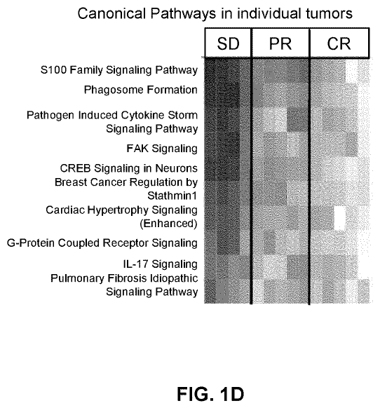

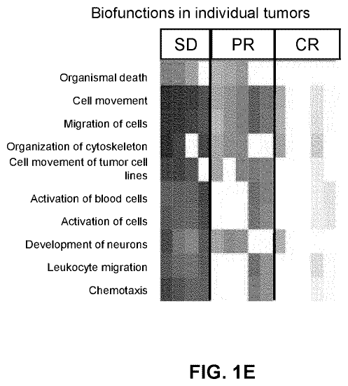

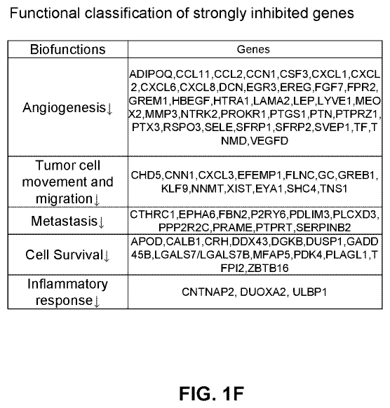

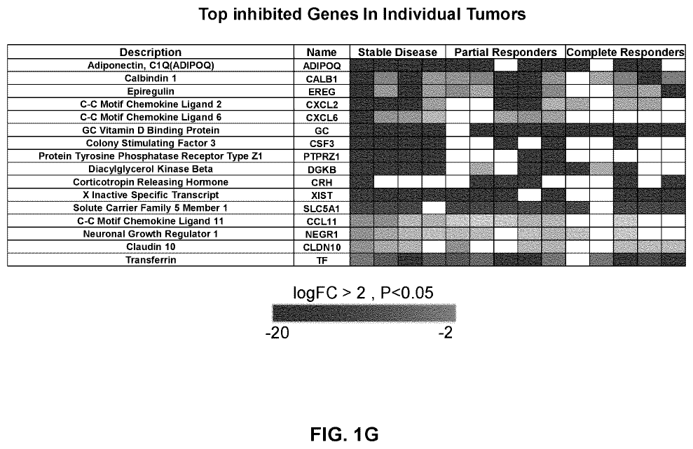

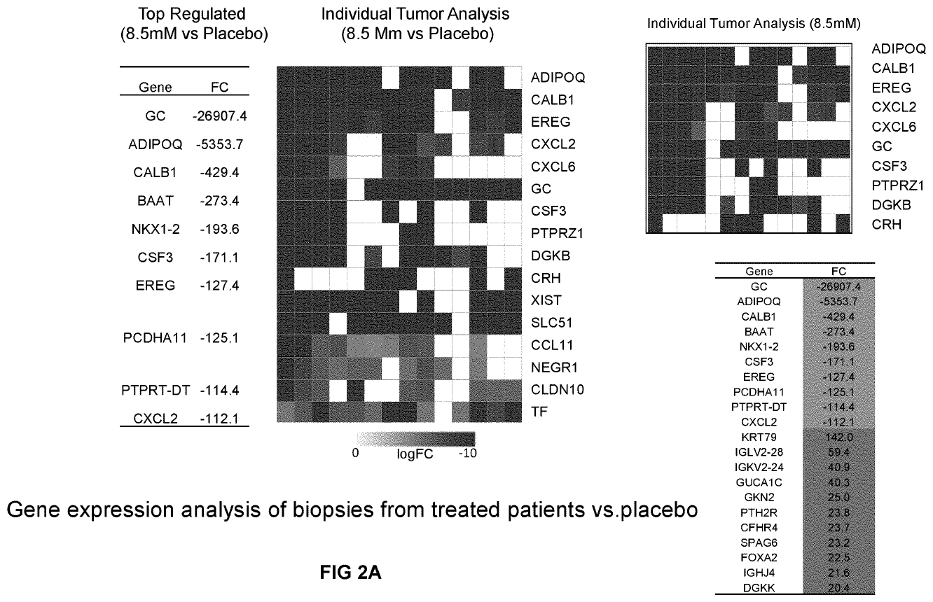

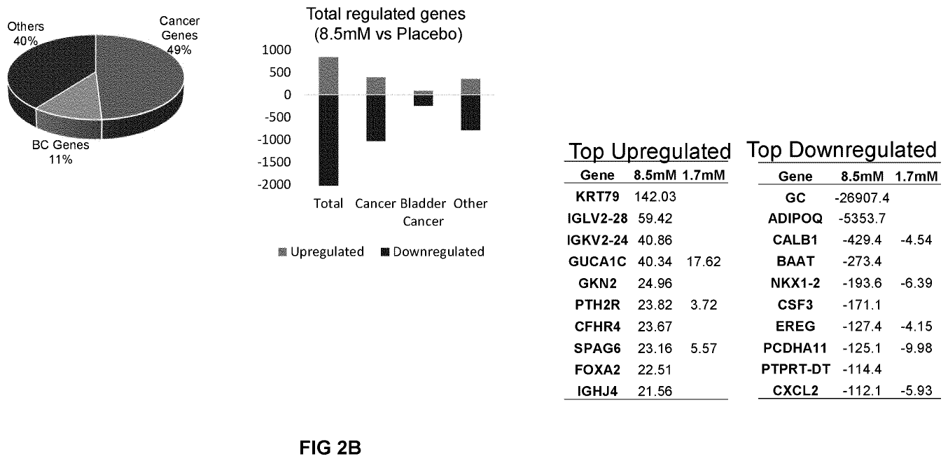

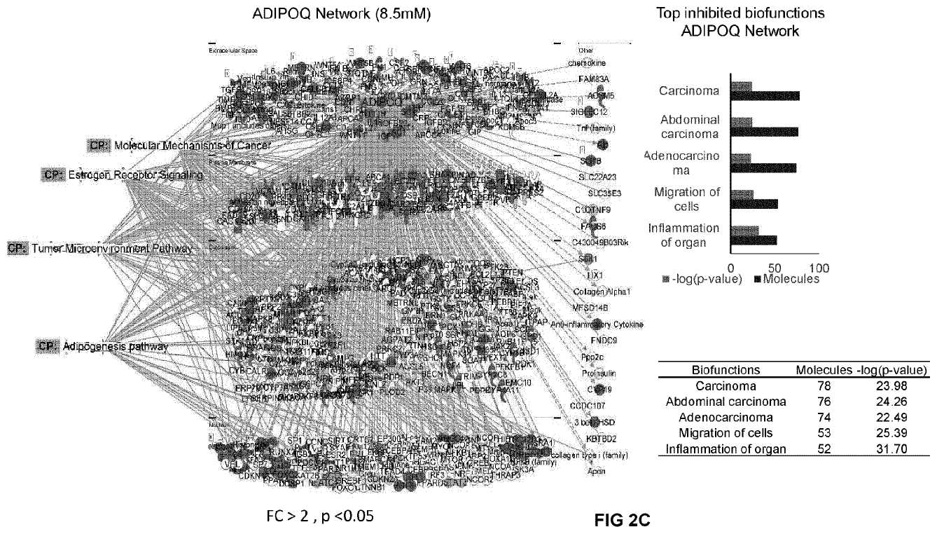

Shed cells were assigned a pathology score as per the Paris classification [20, 21]. Superficial cell shedding and tumor fragmentation was also detected by histopathology of biopsy specimens obtained at surgery (FIG. 2f).

### Alpha1-Oleate Uptake by Tumor Cells and Tissues.

Alpha1-oleate was rapidly internalized by tumor tissue after intra-vesical alpha1-oleate instillation.

Tumor uptake of alpha1-oleate was further quantified in tissue biopsies obtained at TURB.

### DNA Strand Breaks.

Double strand DNA breaks were quantified by TUNEL staining, comparing shed cells post alpha1-oleate instillation, to the pre-instillation samples. A marked increase in TUNEL staining was detected post-instillation in all treated patients.

### Gene Expression Analysis.

The tumor response was characterized in greater detail by sequencing of RNA extracted from tumor tissue obtained at TURB and from biopsies obtained from adjacent tissue with a healthy appearance.

Heat map analysis identified over 3500 genes as being regulated. Functional analysis of the regulated common genes predicted a decrease in cancer-related functions including angiogenesis, tumor cell migration and tumor invasion.

Pathway analysis further confirmed the strong inhibitory effect on cancer related genes and pathways, including the tumor microenvironment, breast cancer and bladder cancer related genes. In addition, the innate immune response was strongly down-regulated, affecting cytokine storm signaling and G protein coupled receptor signaling. Downregulation of chemokines and cytokines including CCL2, CCL11, CCL20, CXCL1, CXCL2 and CXCL12 and IL1B and IL17D were common.

Genes regulating the tumor microenvironment pathway predominated among the cancer related genes, including vascular endothelial growth factors D (VEGFD), transforming growth factor beta genes (TGFB2 and TGFB3), fibroblast growth factors (FGF1, FGF5, FGF7 and FGF10) and insulin like growth factor 1 (IGF1), and were downregulated.

### Bladder Cancer Genes

Bladder cancer related genes were strongly inhibited. Early growth response 3 (EGR3), a VEGF dependent angiogenesis biomarker, was strongly downregulated and genes previously reported to be abundantly expressed in bladder cancer subtypes were regulated, including Protein Kinases Alpha kinase 2 (ALPK2), Platelet derived growth factor receptor beta (PDGFRB) and Protein kinase cGMP-dependent 1 (PRKG1). Transcription factors including the bladder cancer associated Twist family of transcription factor 2 (TWIST2), FOS like 1 (FOSL1) and Zinc finger E-box binding homeobox 1 (ZEB1), markers of epithelial mesenchymal transition and metastasis, were also strongly downregulated.

### Treatment Effect on Individual Tumors

Individual tumors in patients treated with alpha1-oleate were defined as complete responders (CR), if the tumor was removed, partial responders (PR) if the tumor size was reduced, stable disease if the tumor size was unchanged (SD) or as non-responders, if the tumor size increased (Cut off=25% change in tumor size) (FIG. 1a). The effects of alpha1-oleate on gene expression were further examined for individual patients and tumors, using urine samples, and differences were detected relative to the clinical treatment response of each tumor. By Principal component analysis (PCA FIG. 1b) the majority of responding tumors formed a cluster with a similar overall gene expression profile. All stable disease (SD) or non-responding tumors were clustered distantly from this cluster, suggesting a strong effect of alpha1-oleate treatment on these tumors and a difference compared to the remaining group. Consistent with the PCA analysis, biofunction and pathway analysis also showed quantitative differences defined by the z-score intensities, between the responders and the non-responders (FIG. 1c,d).

A group of 297 genes was strongly down-regulated in all treated tumors (log FC≤−0.10 FIG. 1e). Biofunction analysis identified these genes as associated with angiogenesis, tumor cell movement, migration, survival and metastasis, and 31/297 inhibited genes were bladder cancer related, including PRAME nuclear receptor transcriptional regulator (PRAME) and Protein tyrosine phosphatase receptor type T (PTPRT), which were strongly inhibited (log FC≤−10). Similarly, bladder cancer genes previously reported to play important role in angiogenesis were inhibited (log F≤10), including C-X-C motif chemokine ligand 2 (CXCL2), Neurotrophic receptor tyrosine kinas (NTRK2), as well as Protein tyrosine phosphatase receptor type Z1 (PTPRZ1) and Transferrin (TF) (FIG. 1f). The strong inhibition of these molecules across tumors suggested that alpha1-oleate affected all tumors irrespective of their clinical response category.

### SUMMARY

Selecting relevant treatment end points is especially challenging for conditions where cure is elusive. New molecular tools may offer increased resolution of the disease response, to complement clinical observation and understand the potential of new drug candidates. This study used a combination of clinical and molecular tools to define the tumor response to alpha1-oleate in patients with NMIBC. In addition to the evaluation of the change in tumor size, the tumor response was defined by the shedding of tumor cells and fragments into the urine, by the uptake of alpha1-oleate by the tumor and by evidence of tumor cell specific apoptosis. Gene expression analysis of tumor RNA further identified interesting differences between the groups defined as CR, PR and NR by visual inspection. Using tumor cell shedding, apoptosis and inhibition of cancer related genes as end points all patients in this study were definable as responders to alpha1-oleate.

### Materials and Methods

### Peptide Synthesis and Alpha1-Oleate Complex Generation

Peptide synthesis and the preparation of the investigational product were performed under GMP conditions and the complex was diluted in PBS to the final concentration. The placebo group received PBS (sodium chloride, potassium chloride, sodium- and potassium phosphate and water for injection), which was identical in appearance to the active treatment.

### Study Protocol

After diagnosis and informed consent, the subjects received intra-vesical instillations of either alpha1-oleate or placebo on six occasions during one month preceding a scheduled transurethral resection (TURB). A safety follow-up was performed 52 days after the first instillation.

### Primary Endpoints

Safety as Adverse Events profile (time frame: from signing of informed consent (day 1) and until end of study (day 52)): Incidence of adverse events and classification in terms of severity, causality and outcome.
Efficacy as cell shedding (time frame: days 1 to 22): change in cell shedding into urine (number of epithelial cells per mL of urine).
Change from baseline in characteristics of papillary tumors (time Frame: prior to treatment (baseline) and on day 30, in connection with scheduled surgery): the bladder tumors are characterized by in vivo imaging during examination by cystoscopy.

### Secondary Endpoints

Histopathology scoring of the tumor using established parameters for scoring of Grade and Stage/Invasiveness.
Urine cytology examined before and after instillation, using the Paris scoring system.
Uptake of alpha1-oleate by tumor cells, defined by staining with specific antibodies.
Tissue apoptotic response to alpha1-oleate, defined by TUNEL staining.
Tumor response to alpha1-oleate, defined by RNA sequence analysis.
Proteomic analysis of markers in urine was not completed.
Long-term effects of the study treatment have not been evaluated.

### Adverse Events Profile

(AEs) were collected from the signing of the informed consent form until the end of the study (FU1 Visit, day 52). All diagnoses, symptom(s), sign(s) or finding(s) with a start date after first dose of study drug were recorded as AEs or severe AEs (SAEs). (S)AEs related to the study procedure were coded during the course of the trial according to MedDRA by preferred terms and primary system organ class. All adverse events recorded during the course of the trial were included in the subject data listings and an overall summary of the number (percentage) of subjects with any treatment emergent (S)AEs, premature discontinuations from the trial due to AEs, treatment related AEs and (S)AEs was constructed. The number of subjects experiencing each type of adverse events was tabulated regardless of the number of times each adverse event was reported by each subject. The severity of each type of adverse event was also tabulated and graded as the most severe recording for that adverse event.

### Cell Shedding

To quantify the shedding of cells and cell clusters into the urine, samples were obtained from each patient prior to and after each instillation of alpha1-oleate or placebo (Visit 1-Visit 6). Cell shedding was quantified by counting the total number of epithelial cells in a unit of uncentrifuged urine under light microscopy, using a hemocytometer chamber. Changes in cell shedding were quantified at each visit, by comparing cell numbers in samples obtained before and after each instillation. The cell clusters were scored based on the examination of these samples by an experienced pathologist on a range of 0-2 where 0=no clusters and 2=the highest number of clusters.

### Characteristics of Papillary Tumors, Tumor Size

To examine if the alpha1-oleate treatment affects tumor size, all included subjects underwent outpatient cystoscopy at Visit 0. Tumors were reexamined at Visit 7, prior to scheduled surgery. High quality photographs were collected endoscopically, using a flexible cystoscope (Olympus) before being removed by TURB according to EAU Guideline recommendations [23]. Changes in tumor size were evaluated intra-individually, using paired images.

### Histopathology Scoring

Tumor biopsies, collected at the time of surgery were evaluated by histopathology, using established parameters for scoring of Grade and Stage/Invasiveness. Tissue samples were analyzed by a designated study uro-pathologist. Both grading classifications (WHO 1973 and 2004/2016) were used. Biopsies from healthy tissue areas distant from the tumor were collected for comparison.

### Urine Cytology

Urine cells were centrifuged onto L-lysine coated microscope slides (Cytospin 3, Shandon) at 113 g for 5 minutes, fixed and stored at room temperature until further analyses. Urinary cytology was evaluated using the Paris System for Reporting Urinary Cytology 2016 and defined as: 1. No diagnosis/unsatisfactory. 2. Negative for high-grade urothelial carcinoma. 3. Atypical urothelial cells present. 4. Suspicious for high-grade urothelial carcinoma. 5. High grade urothelial carcinoma. 6. Low grade urothelial neoplasm. 7. Other positive for malignancies and miscellaneous lesions.

### Immunohistochemistry

Alpha1-oleate uptake by tumor cells was quantified by staining with specific antibodies. Cells on cytospin-slides were washed (TBS, 10 min), permeabilized (0.25% TritonX-100 in TBS, 20 min, room temperature) and blocked (5% normal goat serum in TBS, 1 h, room temperature) before addition of rabbit polyclonal anti-human alpha lactalbumin antibodies (1:50 in 5% normal goat serum at 4° C., overnight, Mybiosource, Cat #MBS175270). Slides were washed (TBS, 2×5 min) and stained with Alexa-568 labeled secondary antibody (1:200, 1 h, room temperature, ThermoFisher). The nucleus was counterstained using DRAQ5 (1:1000, 15 min) before a final wash (2×5 min in TBS). Slides were mounted (Fluoromount aqueous mounting media), before capturing images by laser scanning confocal microscopy (Carl Zeiss). Fluorescence intensity was quantified by ImageJ and net fluorescence calculated after subtraction of the secondary antibody background.

For other markers, biopsy samples were first deparaffinized in xylene, rehydrated with reduced ethanol concentrations (100%, 95%, 70% and 50%) and then washed with deionized water. The slides were then immerged in target retrieval solution (Dako, 51699) and boiled for 20 minutes, followed by 30 minutes permeabilization with 0.25% Triton in PBS at room temperature. A blocking solution consisting in 5% goat serum in PBS was added on the sections for 1 h at room temperature, before adding the primary antibodies (rabbit polyclonal anti-human alpha-lactalbumin antibody—Mybiosource, Cat #MBS175270.

The slides were then washed with 0.025% PBS-T and stained with goat anti-rabbit Alexa Fluor-488 (Invitrogen, A11034) or goat anti-rabbit Alexa Fluor-568 (Invitrogen, A11036) secondary antibodies (1:200 for 1 h at room temperature,). The nuclei were counterstained with DAPI for 15 minutes before final wash in PBS, and then the slides were mounted with Fluoromount aqueous mounting media (Sigma, F4680). After drying, images were captured with a confocal microscope (Zeiss LSM 900) or Hamamatzu Nanozoomer scanner and fluorescence intensity was quantified by ImageJ.

### Apoptosis Detected by TUNEL Staining

DNA fragmentation was detected using the terminal deoxynucleotidyl transferase dUTP nick end-labelling (TUNEL) assay (Click-iT TUNEL Alexa Fluor 488 imaging assay kit, ThermoFisher). Tissue sections were de-paraffinized with xylene followed by serial dehydration with ethanol (100, 95, 75 and 50%). Dehydrated sections were fixed (4% PFA, 15 min), permeabilized (DNase-free Protease K solution 20 μg/mL, 15 min) and incubated with TUNEL reaction mixture containing TdT for 60 min at 37° C. After the TUNEL reaction, sections were incubated with Click-iT reaction mixture (30 min, 37° C.). Sections were counterstained with DAPI (1 μg/mL, 5 minutes), mounted in Fluoromount aqueous mounting media, and analyzed by fluorescence microscopy (Zeiss). Fluorescence intensities were quantified by ImageJ and net mean fluorescence intensity calculated after subtraction of background fluorescence.

### RNA Sequence Analysis of Tissue Biopsies

RNA was extracted from tissues stabilized in RNAlater using the AllPrep DNA/RNA/miRNA Universal Kit. Disruption was in the TissueLyser system and CK28 Precellys tubes and by homogenization in the QIAshredder homogenizer. The quantity and quality of the RNA samples were evaluated using NanoDrop and Agilent 2100 Bioanalyzer. RNA samples were prepared by Illumina TruSeq Stranded mRNA Library Prep Kit (20020594), and libraries were multiplexed and sequenced using NextSeq 500/550 High Output Kits (v2.5 2×75 Cycles) with an average of 22 million reads per sample. Raw sequencing data was demultiplexed using bcl2fastq (version 2.18) and RSEM (1.3) was used for abundance estimation using the human genome release 37/Ensemble 75. Samples were thoroughly QCed and visualized using dimensionality reduction (i.e PCA), MA-plots as well as RNA-seq intrinsic biases (such as GC bias, transcriptome complexity and alignment quality). Differential expression analysis was performed using R (version 3.4) and the packages limma and DESeq2. Fold change were calculated by comparing tumors in the treated to the placebo group. Relative expression levels were analyzed and genes with an absolute fold change >2.0 and P<0.05 were considered as differentially expressed. Heat-maps were constructed using the Gitools 2.1.1 software. Differentially expressed genes were functionally characterized using the Ingenuity Pathway Analysis version 57662101 (IPA, Qiagen) software.

### Statistical Analysis

For efficacy, the sample size was based on analysis of change in tumor cells assessed from a previous study⁶. A sample size of 20 patients per group was deemed suitable to achieve criterion for significance (alpha) 0.05 and power 90% using the paired samples 1-tailed t-test. The null hypothesis is H₀: mean change in cell shedding=0 and the alternative hypothesis is H_{A}: mean change in cell shedding >0. The Gaussian distribution was determined by the D'agostino & Pearson normality test. For data following a Gaussian distribution, student t-tests were used. Other data sets were analyzed by Mann-Whitney U-test. Correlations were determined by Spearman correlation. Kinetic data was analyzed using the repeated measures 2-way ANOVA test. All statistical analysis was done by using Prism version 6.02 (GraphPad Software Inc.). P values<0.05 were considered as statistically significant. All images were created by the study team.

### Chemicals

Sodium oleate (Sigma-Aldrich, Cat #07501), Click-iT TUNEL Alexa Fluor 488 imaging assay kit (ThermoFisher Scientific Cat #C10245), DRAQ5 (Abcam, Cat #ab108410), Fluoromount (Sigma-Aldrich, Cat #F4680), DNA/RNA/miRNA Universal Kit (Qiagen, Cat #80224),

### Preparation of BAMLET and Alpha1-Oleate

The BAMLET complex was made by mixing bovine alpha-lactalbumin (Sigma, Cat #L5385) with oleic acid (Sigma, Cat #01008). Alpha1 was synthesized using Fmoc solid phase chemistry (Mimotopes). The alpha1 sequence is: aa 1-39 Ac-KQFTKAELSQLLKDIDGYGGIA-LPELIATMFHTSGYDTQ-OH.

### Transcriptomic Analysis

Approximately 5 mg of tissue was homogenized using a Tissuelyser (Qiagen) and total RNA was extracted using the RNeasy kit (Qiagen), amplified using a GeneChip 3′IVT Express Kit, hybridized onto Mouse Genome 430 PM array strips, and scanned using the GeneAtlas system (Affymetrix). Data was normalized using Robust Multi Average implemented in the Transcriptome Analysis Console software (v.4.0.1.36, Applied Biosystems, ThermoFisher Scientific). Relative expression was analyzed by ANOVA using the empirical Bayes method, and genes with an absolute fold change >1.5 or 2.0 were considered differentially expressed. Heat maps were constructed using Graphpad Prism 9 and differentially expressed genes were analyzed using Ingenuity Pathway Analysis software (IPA, Qiagen).

### Involvement of Adiponectin and its Receptors ADIPOR1 and ADIPOR2 in Alpha1-Hamlet's Tumor-Killing Function

### BACKGROUND

The main function of adipose tissue is to store energy, in the form of fat, and comprises 20-25% of the total body weight in healthy individuals. Adipose tissue cells (adipocytes) secrete bioactive molecules called adipocytokines (also called adipokines). The most abundantly secreted adipocytokine is Adiponectin (APN). APN in humans is 244 amino acids and 28 kDa. Circulating APN oligomers exist as trimers (Low molecular weight), hexamers (Middle molecule weight) and multimer (High molecular weight) form, with the latter being the most biologically active form. APN also exists in very small quantities in the plasma in a globular form (as a result of proteolysis).

Adiponectin is a well-known homeostatic factor for regulating glucose levels, lipid metabolism, and insulin sensitivity through its anti-inflammatory, anti-fibrotic, and antioxidant effects. All these metabolic processes are mediated via three receptors, AdipoR1, AdipoR2, and T-cadherin. The AMPK pathway and the PPAR pathway are activated via adiponectin binding to AdipoR1 and AdipoR2 respectively. AdipoR1 and AdipoR2 contain seven transmembrane domains with high structural homology as well as an intracellular N-terminus and an extracellular C-terminus, opposite to that of the classical G-Protein coupled receptors (GPCRs).

### Investigation

In a clinical study from an ongoing randomized, placebo-controlled phase II trial in patients with non-muscle invasive bladder cancer (NMIBC), treatment with the tumor-killing peptide-lipid complex Alpha1-Hamlet, by instillations to the patients' bladders, has shown promising therapeutic effects. Tumor tissue samples of patients (obtained post-treatment at visit number 6) were analyzed. The gene expression profiling analysis of these samples revealed that ADIPOQ gene, which encodes for the adiponectin protein, was also the second most regulated gene, with a highly significant downregulation (FC=−5353.7) in the 8.5 mM Alpha1-H treated patients compared to placebo treated patients. the regulatory effects of top regulated molecules using network and pathway analyses (FIG. 2).

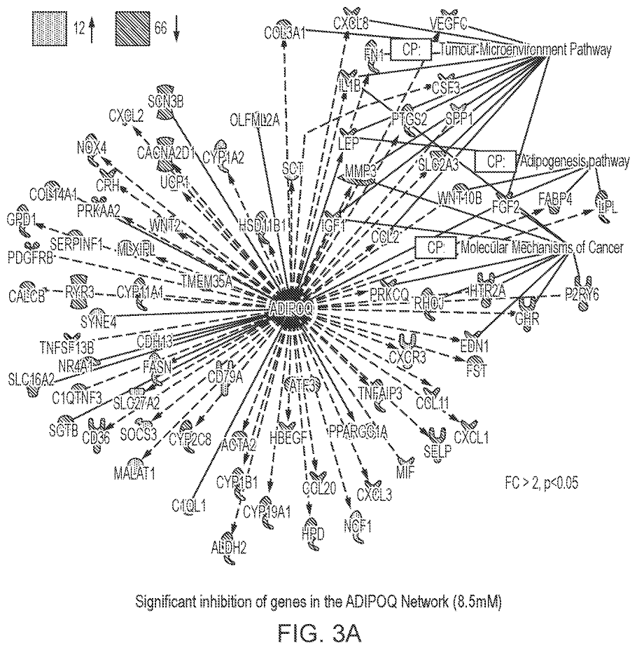

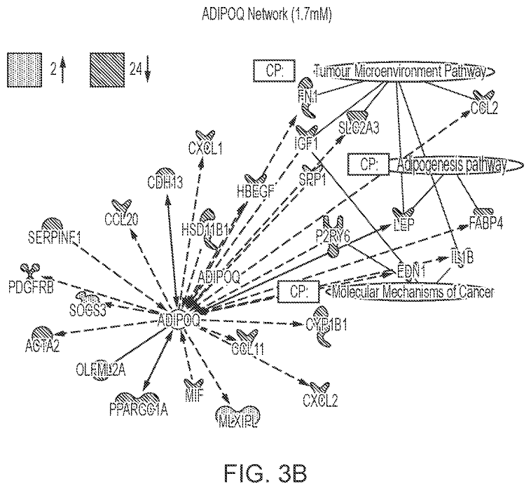

Network analysis of ADIPOQ showed that 78 ADIPOQ dependent molecules were strongly regulated out of which 66 were downregulated while 12 were upregulated. Interestingly, ADIPOQ and its associated molecules showed regulation of several pathways including molecular mechanism of cancer, tumor microenvironment and adipogenesis signaling. (FIG. 3)

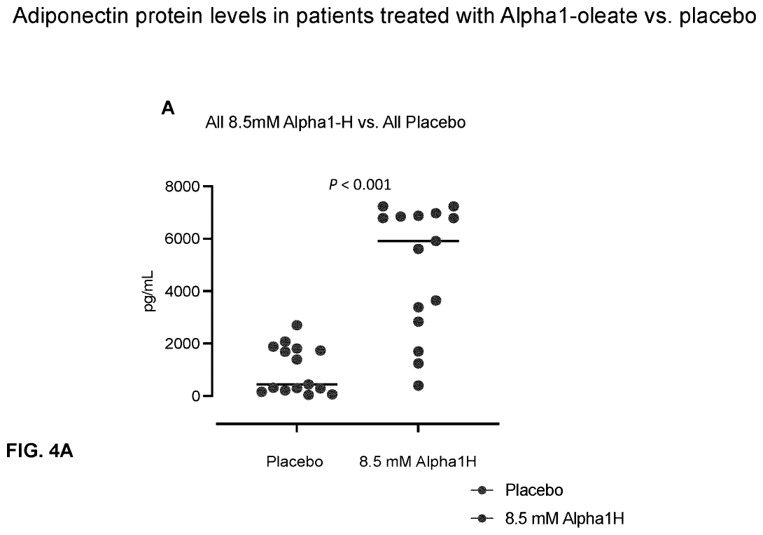

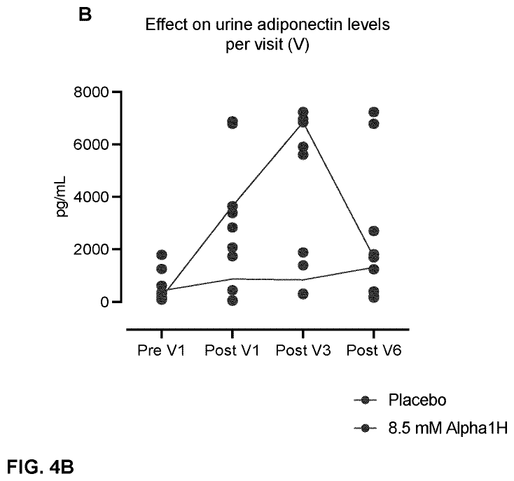

To investigate levels of adiponectin protein in the urine samples of patients, an Adiponectin-ELISA test (Proteintech) was performed. The signal (absorbance) values were measured at 480 nm and interpolated to concentration (pg/mL) values using a linear regression statistical analysis. Measured urine adiponectin levels are significantly higher in Alpha1-oleate treated patients vs placebo (P<0.001), Mann-Whitney T-test (FIG. 4). Graph showing urine adiponectin levels in both groups, with samples taken pre Visit 1, post Visit 1, Post Visit 3 and Post Visit 6. Line show change in median urine adiponectin levels (FIG. 4). There was statistically significant positive correlation between number of cells (shed) in urine samples and measure adiponectin levels (P=0,002), using linear regression (FIG. 4). Statistical analysis was done in GraphPad Prism.

### The Adiponectin Receptors

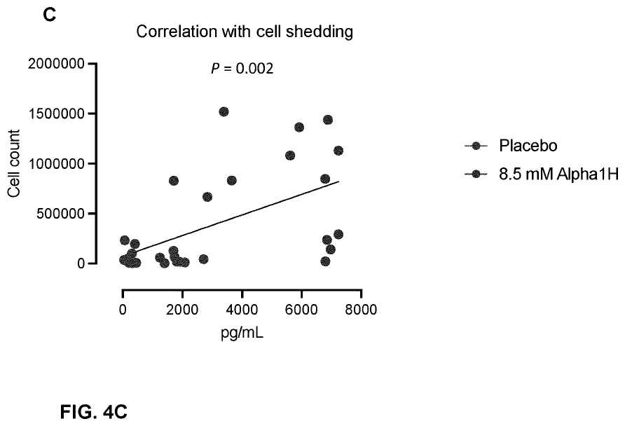

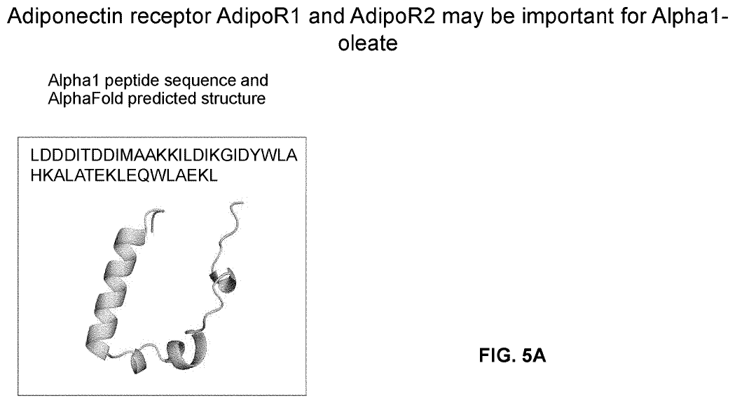

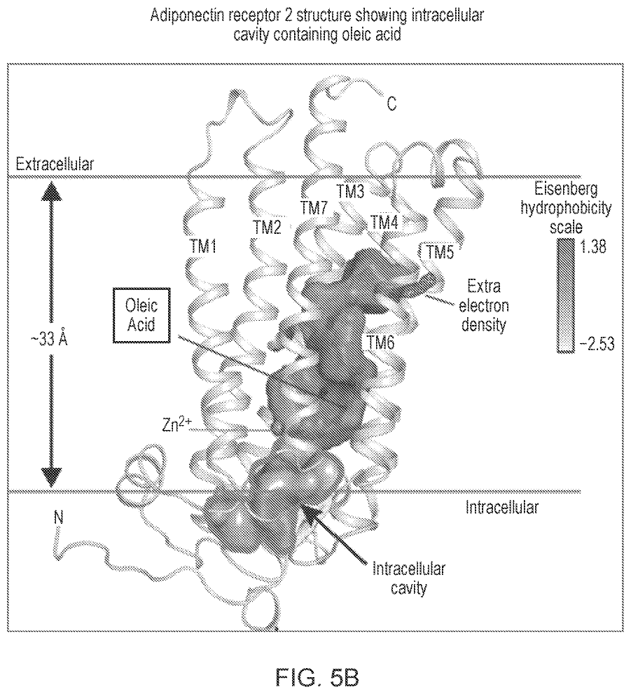

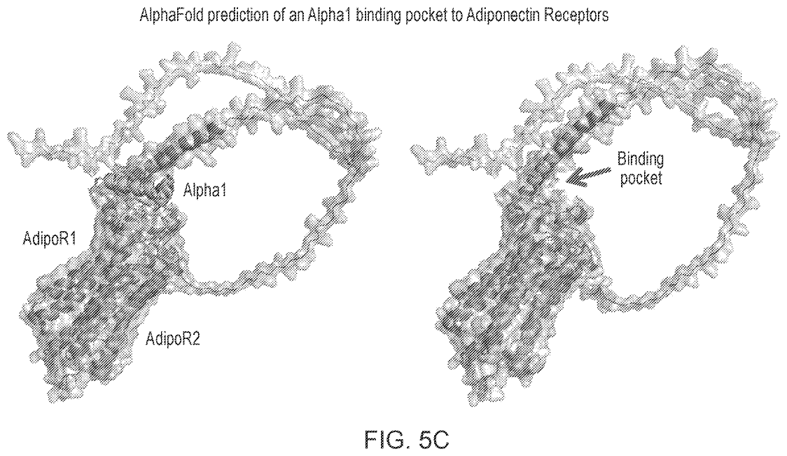

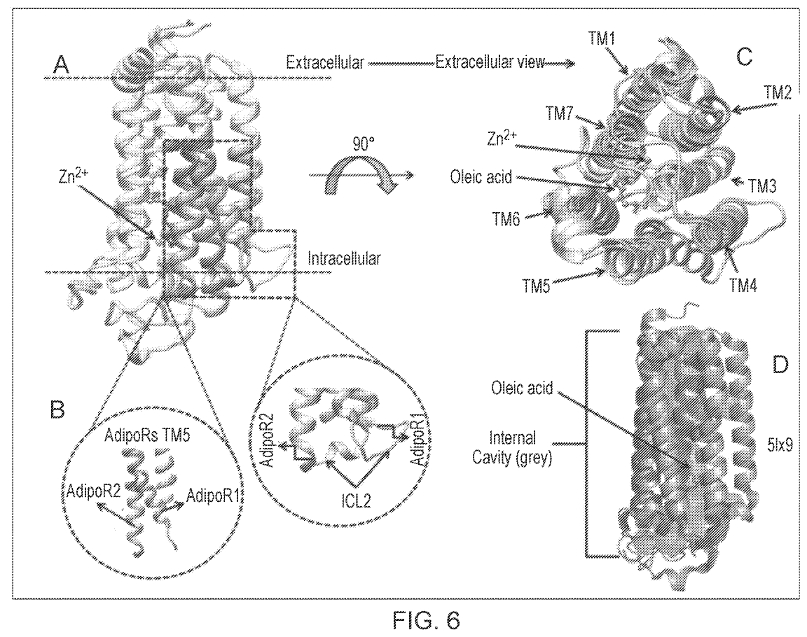

Previously reported crystal structures of the AdipoR1 and AdipoR2 proteins interestingly show that the major cavity in the protein structure can be occupied by oleic acid. AdipoR1 and AdipoR2 also have an adiponectin-independent function of maintaining membrane fluidity in many types of human cells. The inventors find this highly useful information in learning about Alpha1-H's tumor-killing mechanism, given the fact that the complex has an oleic acid component and could therefore potentially interact with the AdipoR1 and AdipoR2 receptors at the membrane level. Furthermore, Alpha1-oleate is highly membrane active, suggesting that the combined effects of alpha1-oleate and the adiponectin receptors may be essential to initiate the extensive tumor cell to alpha1-oleate, that leads to tumor cell death. (FIG. 5).

In silico modeling was used to predict if the alpha1 peptide shows affinity for the AdipoQ receptors. The Alphafold software, which predicts protein-protein interactions, suggested a common binding pocket region for Alpha1 (shown in green) near the intracellular N-terminal domain of both adiponectin receptors (shown in cyan and orange) (FIG. 5).

### CLAUSES

1. A method of predicting the outcome of treatment of cancer in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of cancer genes in the subject following administration of the complex, and b) correlating the regulation of the genes to the outcome of the treatment, wherein a positive outcome of treatment is inversely related to the level of regulation of those genes.

2. The method of clause 1, wherein the cancer is found in one or more of the nasal passage, the GI tract (e.g., in one or more of the oral cavity, the stomach, the colon, the bowel), the central nervous system (e.g., in the brain, the spine), the lung, the kidney, the vagina, the bladder, the liver, the skin, the breast, the prostate and/or the ovary.

3. The method of clause 2, wherein the cancer is bladder cancer.

4. A method of predicting the outcome of treatment of a metabolic-related condition in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of cancer genes in the subject following administration of the complex, and b) correlating the regulation of the genes to the outcome of the treatment, wherein a positive outcome of treatment is inversely related to the level of regulation of those genes.

5. The method of any preceding clause, wherein the method comprises assessing the level of cancer gene regulation by measuring a marker of cancer gene regulation in a sample obtained from the subject.

6. The method of clause 5, wherein the sample is a sample of bodily fluid.

7. The method of clause 6, wherein the sample is a urine sample.

8. The method of any of clauses 5 to 7, wherein the marker is RNA or DNA.

9. The method of clause 8, wherein the marker is RNA.

10. The method of any preceding clause, wherein the cancer gene or genes are genes regulated by the complex.

11. The method of any preceding clause, wherein the cancer gene or genes comprise one, two, three, four, five, six, seven, eight, nine, ten, 15, 20 or 25 or more cancer genes, the genes comprising one, two, three, four, five, six, seven, eight, nine, ten or more genes selected from ADIPOQ, CALB1, EREG, CXCL2, CXCL6, GC, CSF3, PTPRZ1, DGKB, CRH, XIST, SLC5A1, CCL11, NEGR1, CLDN10, TF.

12. The method of clause 11, wherein the cancer gene or genes comprise ADIPOQ.

13. The method of clause 11 or 12, wherein the cancer gene or genes comprise CALB1.

14. The method of any of clauses 11, 12 and 13, wherein the cancer gene or genes comprise EREG.

15. The method of any of clauses 11 to 14, wherein the cancer gene or genes comprise CXCL2.

16. The method of any of clauses 11 to 15, wherein the cancer gene or genes comprise CXCL6.

17. The method of any of clauses 11 to 16, wherein the cancer gene or genes comprise GC.

18. The method of any of clauses 11 to 17, wherein the cancer gene or genes comprise CSF3.

19. The method of any of clauses 11 to 18, wherein the cancer gene or genes comprise PTPRZ1.

20. The method of any of clauses 11 to 19, wherein the cancer gene or genes comprise DGKB.

21. The method of any of clauses 11 to 20, wherein the cancer gene or genes comprise CRH.

22. The method of any of clauses 11 to 21, wherein the cancer gene or genes comprise XIST.

23. The method of any of clauses 11 to 22, wherein the cancer gene or genes comprise SLC5A1.

24. The method of any of clauses 11 to 23, wherein the cancer gene or genes comprise CCL11.

25. The method of any of clauses 11 to 24, wherein the cancer gene or genes comprise NEGR1.

26. The method of any of clauses 11 to 25, wherein the cancer gene or genes comprise CLDN10.

27. The method of any of clauses 11 to 26, wherein the cancer gene or genes comprise TF.

28. A method of treating cancer, comprising administering a therapeutic amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to a subject; assessing the regulation of cancer genes in the subject following administration of the complex and correlating the regulation of the genes to the outcome of the treatment, using the method of any one of clauses 1 to 3 or 5 to 27, and determining and/or a further course of treatment.

29. A method of treating a me, comprising administering a therapeutic amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to a subject; assessing the regulation of cancer genes in the subject following administration of the complex and correlating the regulation of the genes to the outcome of the treatment, using the method of any one of clauses 4 to 27, and determining and/or a further course of treatment.

### REFERENCES

1. Hakansson, A., et al., Apoptosis induced by a human milk protein. Proc Natl Acad Sci USA, 1995. 92(17): p. 8064-8.
2. Svensson, M., et al., Conversion of alpha-lactalbumin to a protein inducing apoptosis. Proc Natl Acad Sci USA, 2000. 97(8): p. 4221-6.
3. Pettersson-Kastberg, J., et al., alpha-Lactalbumin, engineered to be nonnative and inactive, kills tumor cells when in complex with oleic acid: a new biological function resulting from partial unfolding. J Mol Biol, 2009. 394(5): p. 994-1010.
4. Fischer, W., et al., Human alpha-lactalbumin made lethal to tumor cells (HAMLET) kills human glioblastoma cells in brain xenografts by an apoptosis-like mechanism and prolongs survival. Cancer Res, 2004. 64(6): p. 2105-12.
5. Gustafsson, L., et al., Treatment of skin papillomas with topical alpha-lactalbumin-oleic acid. N Engl J Med, 2004. 350(26): p. 2663-72.
6. Mossberg, A. K., et al., HAMLET treatment delays bladder cancer development. J. Urol, 2010. 183(4): p. 1590-7.
7. Puthia, M., et al., Prevention and treatment of colon cancer by peroral administration of HAMLET (human alpha-lactalbumin made lethal to tumour cells). Gut, 2014. 63(1): p. 131-42.
8. Mossberg, A. K., et al., Bladder cancers respond to intravesical instillation of HAMLET (human alpha-lactalbumin made lethal to tumor cells). Int J Cancer, 2007. 121(6): p. 1352-9.
9. Brisuda, A., et al., Bladder cancer therapy using a conformationally fluid tumoricidal peptide complex. Nat Commun, 2021. 12(1): p. 3427.
10. Ho, C. S., et al., Low resolution solution structure of HAMLET and the importance of its alpha-domains in tumoricidal activity. PLoS One, 2012. 7(12): p. e53051.
11. Antoni, S., et al., Bladder Cancer Incidence and Mortality: A Global Overview and Recent Trends. Eur Urol, 2017. 71(1): p. 96-108.
12. van Rhij n, B. W., et al., Recurrence and progression of disease in non-muscle-invasive bladder cancer: from epidemiology to treatment strategy. Eur Urol, 2009. 56(3): p. 430-42.
13. Hu, Q., et al., Recent advances of cocktail chemotherapy by combination drug delivery systems. Adv Drug Deliv Rev, 2016. 98: p. 19-34.
14. Sharma, P. and J. P. Allison, Immune checkpoint targeting in cancer therapy: toward combination strategies with curative potential. Cell, 2015. 161(2): p. 205-14.
15. Ourfali, S., et al., Recurrence Rate and Cost Consequence of the Shortage of Bacillus Calmette-Guerin Connaught Strain for Bladder Cancer Patients. Eur Urol Focus, 2021. 7(1): p. 111-116.
16. de Jong, J. J., et al., Hyperthermic Intravesical Chemotherapy for BCG Unresponsive Non-Muscle Invasive Bladder Cancer Patients. Bladder Cancer, 2018. 4(4): p. 395-401.
17. Colombo, R., et al., Multicentric study comparing intravesical chemotherapy alone and with local microwave hyperthermia for prophylaxis of recurrence of superficial transitional cell carcinoma. J Clin Oncol, 2003. 21(23): p. 4270-6.
18. Arends, T. J., et al., Results of a Randomised Controlled Trial Comparing Intravesical Chemohyperthermia with Mitomycin C Versus Bacillus Calmette-Guerin for Adjuvant Treatment of Patients with Intermediate- and High-risk Non-Muscle-invasive Bladder Cancer. Eur Urol, 2016. 69(6): p. 1046-52.
19. Hien, T. T., et al., Bladder cancer therapy without toxicity—A dose-escalation study of alpha1-oleate. International Journal of Cancer, 2020. 147(9): p. 2479-2492.
20. Barkan, G. A., et al., The Paris System for Reporting Urinary Cytology: The Quest to Develop a Standardized Terminology. Acta Cytol, 2016. 60(3): p. 185-97.
21. VandenBussche, C. J., A review of the Paris system for reporting urinary cytology. Cytopathology, 2016. 27(3): p. 153-6.
22. Gono, K., Narrow Band Imaging: Technology Basis and Research and Development History. Clin Endosc, 2015. 48(6): p. 476-80.
23. Babjuk, M., et al., EAU guidelines on non-muscle-invasive urothelial carcinoma of the bladder: update 2016. Eur. Urol., 2017. 71(3): p. 447-461.

1. A method of predicting the outcome of treatment of cancer in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of cancer genes in the subject following administration of the complex, and b) correlating the regulation of the genes to the outcome of the treatment, wherein a positive outcome of treatment is inversely related to the level of regulation of those genes.

2. The method of claim 1, wherein the cancer is found in one or more of the nasal passage, the GI tract (e.g., in one or more of the oral cavity, the stomach, the colon, the bowel), the central nervous system (e.g., in the brain, the spine), the lung, the kidney, the vagina, the bladder, the liver, the skin, the breast, the prostate and/or the ovary.

3. The method of claim 2, wherein the cancer is bladder cancer.

4. A method of predicting the outcome of treatment of cancer in a subject, said treatment comprising the administration of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to the subject; wherein the method comprises the steps of a) assessing the regulation of ADIPOQ network genes in the subject following administration of the complex, and b) correlating the regulation of the genes to the outcome of the treatment, wherein a positive outcome of treatment is inversely related to the level of regulation of those genes.

5. The method of any preceding claim, wherein the method comprises assessing the level of cancer gene regulation by measuring a marker of cancer gene regulation in a sample obtained from the subject.

6. The method of claim 5, wherein the sample is a sample of bodily fluid.

7. The method of claim 6, wherein the sample is a urine sample.

8. The method of any of claims 5 to 7, wherein the marker is RNA or DNA.

9. The method of claim 8, wherein the marker is RNA.

10. The method of any preceding clause, wherein the cancer gene or genes are genes regulated by the complex.

11. The method of any preceding claim, wherein the cancer gene or genes comprise one, two, three, four, five, six, seven, eight, nine, ten, 15, 20 or 25 or more cancer genes, the genes comprising one, two, three, four, five, six, seven, eight, nine, ten or more genes selected from ADIPOQ, CALB1, EREG, CXCL2, CXCL6, GC, CSF3, PTPRZ1, DGKB, CRH, XIST, SLC5A1, CCL11, NEGR1, CLDN10, TF.

12. A method of treating cancer, comprising administering a therapeutic amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to a subject; assessing the regulation of cancer genes in the subject following administration of the complex and correlating the regulation of the genes to the outcome of the treatment, using the method of any one of claims 1 to 3 or 5 to 11, and determining and/or a further course of treatment.

13. A method of treating a metabolic-related condition, comprising administering a therapeutic amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof to a subject; assessing the regulation of ADIPOQ network genes in the subject following administration of the complex and correlating the regulation of the genes to the outcome of the treatment, using the method of any one of claims 4 to 11, and determining and/or a further course of treatment.
